(19) United States
(12) Patent Application Publication
(10) Pub. No.: US 2014/0011826 A1
(43) Pub. Date: Jan. 9, 2014

(54) METABOTROPIC GLUTAMATE RECEPTOR GROUP I ANTAGONISTS FOR TREATMENT OF ABNORMAL UNION OF TISSUE

(71) Applicant: Monika Bauden, Malmo (SE)

(72) Inventor: Monika Bauden, Malmo (SE)

(21) Appl. No.: 13/984,400

(22) PCT Filed: Feb. 9, 2012
(86) PCT No.: PCT/SE2012/050132

§ 371 Date: Sep. 25, 2013

(30) Foreign Application Priority Data
Feb. 11, 2011 (SE) 1130004-3

(51) Int. Cl. A61K 31/44

(52) U.S. Cl. CPC A61K 31/44
USPC 514/277; 546/350

(57) ABSTRACT

Use of a metabotropic glutamate receptor group I antagonist, or a pharmaceutically acceptable salt, prodrug or an optical isomer thereof, for the manufacture of a medicament for the treatment or the prevention of abnormal union of tissue, is provided. A related method is also provided.

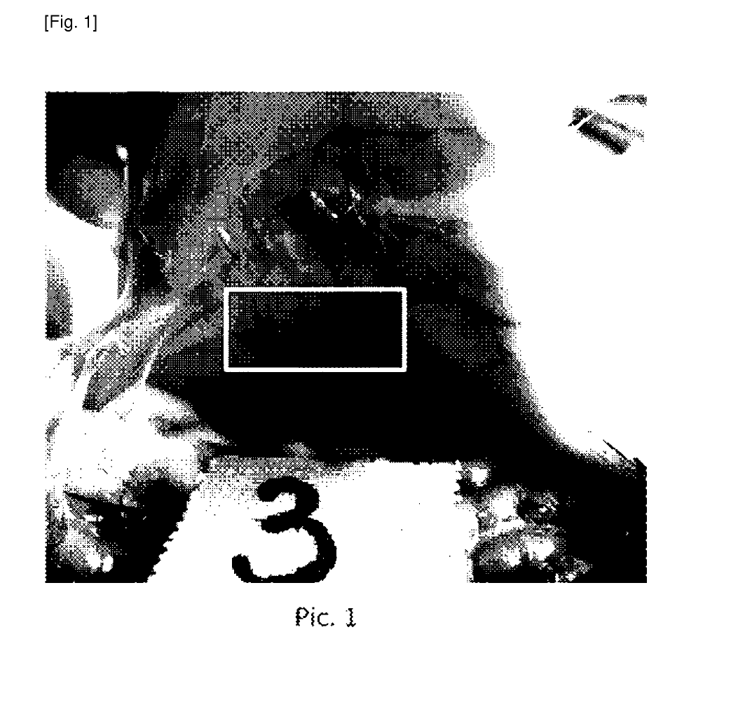

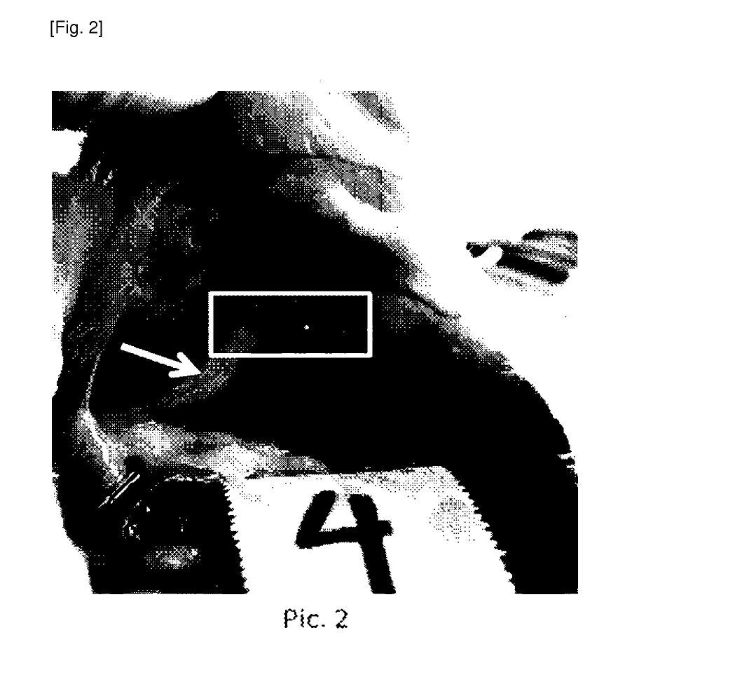

### TECHNICAL FIELD

The present invention relates to the use of metabotropic glutamate receptor group I antagonists for treatment of abnormal union of tissue, in particular intra abdominal adhesions.

### BACKGROUND

Abnormal fibrous bands between organs and tissues in the abdominal cavity are commonly known as intra abdominal adhesions. Adhesions comprise an organized persistent fibrous matrix and constitute an abnormal linkage between adjacent tissues, e.g. two organs or an organ and adjacent tissue, which are normally separated. Peritoneal injury during abdomino-pelvic surgery is a common cause of post-operative intra abdominal adhesions. It has been estimated that 93-100% of patients undergoing abdomino-pelvic interventions develop intra abdominal adhesions. Generally, post-operative adhesions account for 40% of all cases of intestinal obstructions, of which 60-75% involves the small bowels. Ovarian adhesions occur in more than 90% of women after gynecological surgery, being responsible for as many as 20-40% of all cases of secondary female infertility. Hence, adhesions are highly undesired due to the suffering they cause the patient in the form of e.g. pain and the increased risk of secondary medical conditions and disease states as exemplified above. Despite the great medical need, no ideal method for the prevention of adhesion formation is currently available. Current or investigative methods for the prevention of post-operative adhesion formation may be divided into pharmaceutical intervention and physical barriers that mechanically separate tissues as summarized in B. C. Ward et al. Journal of Surgical Research 165, 91-11 (2011). Examples of the former methods include treatments with anticoagulants, fibrinolytics, thromboxane A2 receptor blockers, anti-inflammatories, antihistamines, growth factor inhibitors, epidermal growth factor, ACE inhibitors, angiotensin II receptor blockers, matrix metalloproteinases, immunosuppressives, octreotide, systemic and locally administered antibiotics, taurolidine, phospholipids, collagen inhibitors, medroxyprogesterone and leuprolide acetate, hypoestrogenic environment inducers, aromatase inhibitors, phosphodiesterase 5 inhibitors, methylene blue, catalase, vitamin E, opioids, local anesthetics, sphingosine kinase 1 via adenovirus and neurokinin-1 receptor antagonists. Examples of physical barriers include solid sheets of carboxymethylcellulose and hyaluronic acid, and oxidized sheets of regenerated cellulose.

Glutamate is an excitatory neurotransmitter acting via glutamate receptors (GluRs) including, for example, metabotropic glutamate receptors (mGluR) and N-methyl-D-aspartate receptor (NMDAR). Beside the CNS, accumulating evidence reveal that GluRs are also widely present peripherally within various tissues in both neuronal cells and non-neuronal cells including sensory afferents, the vagus nerve, the immune tissue, the gastrointestinal (GI) tract, the female reproductive system, lymphoid tissues and inflammatory infiltrates, as summarized in S. Santokh et al. Toxicologic Pathology, 29/2, 208-223 (2001). Metabotropic glutamate receptor subtypes (mGluRs) may be subdivided into three groups, group I, group II, and group III, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium. Advances in the elucidation of the neurophysiological roles of metabotropic glutamate receptors in general, and group I in particular, have established these receptors as promising drug targets in the therapy of e.g. acute and chronic neurological and psychiatric disorders, i.e. disorders highly related to the central nervous system. More recently, peripherally occurring mGluR group I receptors have been recognized as promising targets for the development of small molecular drugs for the treatment of e.g. gastrointestinal disorders.

WO2004000316A1 discloses the use of metabotropic glutamate receptor 5 (mGluR5) antagonists for the inhibition of e.g. transient lower esophageal sphincter relaxations and gastro-esophageal reflux disease.

WO2005058361A1 discloses the use of metabotropic glutamate receptor 1 (mGluR1) antagonists for the inhibition of e.g. transient lower esophageal sphincter relaxations and gastro-esophageal reflux disease.

WO2005060965A1 discloses the use of metabotropic glutamate receptor 5 (mGluR5) antagonists for the treatment of irritable bowel syndrome (IBS).

WO2005058323A1 discloses the use of metabotropic glutamate receptor 1 (mGluR1) antagonists for the treatment of irritable bowel syndrome (IBS).

WO2004000316A1, WO2005058361A1, WO2005060965A1 and WO2005058323A1, all relating to intra abdominal conditions and metabotropic glutamate receptor group I antagonists, fail to mention the use of a metabotropic glutamate receptor group I antagonist, i.e. an mGluR1 and/or mGluR5 antagonist, for treatment of abnormal union of tissue such as intra abdominal adhesions.

The treatments for prevention, revocation or reduction of abnormal union of tissue, like e.g. intra abdominal adhesions developed post-operatively, are in many ways insufficient. Hence, novel methods for treatment of abnormal union of tissue are desired. In particular, methods for the prevention of intra abdominal adhesions that occur after surgery are most desired.

### SUMMARY

The present invention seeks to mitigate, alleviate, circumvent or eliminate at least one, such as one or more, of the above-identified deficiencies.

Accordingly there is provided, according to one aspect of the invention, a metabotropic glutamate receptor group I antagonist, or a pharmaceutically acceptable salt, prodrug or an optical isomer thereof, for use in treatment or prevention of abnormal union of tissue.

According to another aspect of the invention, there is provided the use of a metabotropic glutamate receptor group I antagonist, or a pharmaceutically acceptable salt, prodrug or an optical isomer thereof, for the manufacture of a medicament for treatment or prevention of abnormal union of tissue.

According to yet another aspect of the invention, there is provided a method for the treatment or prevention of abnormal union of tissue, wherein an effective amount of a metabotropic glutamate receptor group I antagonist or a pharmaceutically acceptable salt, prodrug or an optical isomer thereof, or an effective amount of a pharmaceutical composition comprising a metabotropic glutamate receptor group I antagonist or a pharmaceutically acceptable salt, prodrug or an optical isomer thereof, is administered to a subject in need of such treatment or prevention.

Further, advantageous features of various embodiments of the invention are defined in the dependent claims and within the detailed description below.

### DETAILED DESCRIPTION

It has surprisingly been found that metabotropic glutamate receptor group I antagonists are useful for the treatment or prevention of abnormal union of tissue. Hence, metabotropic glutamate receptor group I antagonists may be useful for prevention of, for example, intra abdominal adhesions frequently occurring after surgery wherein the peritoneum is pierced. The metabotropic glutamate receptor group I antagonists may be a selective mGluR1 antagonist, a selective mGluR5 antagonist or a dual antagonist of both mGluR1 and mGluR5. A dual antagonist of both mGluR1 and mGluR5 (dual mGluR1/5 antagonist) is an mGluR1 antagonist and an mGluR5 antagonist.

The peritoneum, in similarity to abdominal organs and formations (viscera), is innervated sympathetically and parasympathetically by the vagus nerve and by the splanchnic nerves. Sensory information about the state of the viscera is transmitted by vagal and spinal afferent endings. Without being bound to any theory, the inventor believes that the metabotropic glutamate receptor group I antagonists may minimize e.g. post-operative abdominal adhesions by a reduction of an exaggerated immunologic and/or nociceptive response to surgery, as well as diminishing peripheral and central sensitization. In turn, this may lead to a decreased activation of various cellular mediators responsible for release of pro inflammatory factors that are maintaining the inflammatory process needed for adhesion formation, and thus to minimized adhesion formation. An advantage of metabotropic glutamate receptor group I antagonists for prevention of abnormal union of tissue include the modulatory behavior of the target receptors. Undesired side effects, such as inhibited wound healing after surgery, are thus minimized.

Herein, the term ‘antagonist’ should be understood as including full antagonists, inverse agonists, non-competitive antagonists, uncompetitive antagonist, silent antagonist or competitive antagonists, as well as partial antagonists, whereby a ‘partial antagonist’ should be understood as a compound capable of partially, but not fully, in-activating a metabotropic glutamate receptor group I.

Herein, the term ‘mGluRgIa’ is to be understood as ‘metabotropic glutamate receptor group I antagonist’.

Pictures

Pic. 1 shows the open abdomen of a mGluRgIa treated rat (no. 3) of Example 1, with the scar formed after a 18 mm long sharp incision through the musculoperitoneal tissue on the left lateral abdominal wall (within white square), with no visible adhesions; and

Pic. 2 shows the open abdomen of a vehicle only treated rat (no. 4) of Example 1, with the scar formed after a 18 mm long sharp incision through the musculoperitoneal tissue on the left lateral abdominal wall (within white square), with a clearly visible adhesion extending from the scar (white arrow).

### Embodiments of the Invention

Accordingly there is provided, according to one aspect of the invention, a metabotropic glutamate receptor group I antagonist, or a pharmaceutically acceptable salt or an optical isomer thereof, for use in treatment or prevention of abnormal union of tissue.

According to another aspect of the invention, there is provided the use of a metabotropic glutamate receptor group I antagonist, or a pharmaceutically acceptable salt or an optical isomer thereof, for the manufacture of a medicament for treatment or prevention of abnormal union of tissue.

According to yet another aspect of the invention, there is provided a method for thetreatment or prevention of abnormal union of tissue, wherein an effective amount of a metabotropic glutamate receptor group I antagonist, or an effective amount of a pharmaceutical composition comprising a metabotropic glutamate receptor group I antagonist, is administered to a subject in need of such treatment or prevention.

According to one embodiment, the mGluRgIa may be anmGluR5 antagonist, such as e.g. a mGluR5 antagonist compound with a molecular weight of less than 1000 g/mol, such as less than 500 g/mol.

According to one embodiment, the mGluRgIa may be anmGluR1 antagonist, such as e.g. a mGluR1 antagonist compound with a molecular weight of less than 1000 g/mol, such as less than 500 g/mol.

According to one embodiment, the mGluRgIa may be a dualmGluR1-mGluR5 antagonist, i.e. a compound acting as both a mGluR1 antagonist and as an mGluR5 antagonist. The dual mGluR1-mGluR5 antagonist may be a compound with a molecular weight of less than 1000 g/mol, such as less than 500 g/mol.

According to one embodiment, the mGluRgIa may be the mGluR5 antagonist 2-methyl-6-(phenylethynyl)-pyridine (MPEP). MPEP is either commercially available or may be synthesized according to well-known procedures such as disclosed by K. Sonogashira et al. in Tetrahedron Lett. (1975), 50, 4467-4470.

According to one embodiment, the mGluRgIa may be the mGluR5 antagonist 3-[(2-Methyl-1,3-thiazol-4-yl)ethynyl]pyridine (MTEP). MTEP is described in Cosford N. D. P. et al. J. Med. Chem. 46, 204-206 (2003).

According to one embodiment, the mGluRgIa may be selected from the group of mGluR5 antagonist consisting of SIB-1757, SIB-1893, AFQ056, NPL-2009, AZD2066, ADX10059, AZD2516, ADX48621, STX107 and ADX63365.

According to one embodiment, the mGluRgIa may be selected from the group of mGluR1 antagonist consisting of SYN-119, LY367385, CPCCOEt, Bay 36-7620, JNJ16259685, FTIDC and YM298198.

According to one embodiment, the mGluRgIa may be an mGluR1 antagonist or mGluR5 antagonist disclosed in WO2004014370A2, WO2004014881A2, WO2004014902A2, WO2005080356A1, WO2005080363A1, WO2005080386A1, WO2006014185A1, WO2007021574A1, WO2007021575A2, WO2007130825A2, WO2009051556A1, WO2009054785A1, WO2009054786A1, WO2009054789A1, WO2009054790A1, WO2009054791A1, WO2009054792A1, WO2009054793A1, WO2009054794A1 or WO2010123451A1, herein incorporated by reference.

According to one embodiment, the mGluRgIa may be an mGluR1 antagonist or mGluR5 antagonist disclosed in WO02068417A2, WO2005066155A1, WO2004000316A1, WO2005080379A1, WO2005080397A2, WO2007040982A1, WO2010019100A1, WO2007130820A2, WO2007130821A2, WO2007130822A2, WO2007130823A2, WO2007130824A2, WO2008041075A1, WO2009054787A1, WO9926927A2 or WO2004069813A1, herein incorporated by reference.

According to one embodiment, the mGluRgIa may be an mGluR5 antagonist disclosed in WO02068417A2, such as 3-(2-pyridyl)-5-(3-methoxyphenyl)-1,2,4-oxadiazole, 3-(2-pyridyl)-5-(3,5-dichlorophenyl)-1,2,4-oxadiazole, 3-(2-pyridyl)-5-(3-chlorophenyl)-1,2,4-oxadiazole, 3-(2-pyridyl)-5-(2-chlorophenyl)-1,2,4-oxadiazole, 3-(2-pyridyI)-5-[3-(trifluoromethyl)phenyl]-1,2,4-oxadiazole, 3-(2-pyridyl)-5-(3-methylphenyl)-1,2,4-oxadiazole, 3-(2-pyridyl)-5-(1-naphthyl)-1,2,4-oxadiazole, 3-(2-pyridyl)-5-[3-(trifluoromethoxy)phenyl]-1,2,4-oxadiazole, 3-(2-pyridyl)-5-(2,3-difluorophenyl)-1,2,4-oxadiazole, 3-(2-pyridyl)-5-(2,5-difluorophenyl)-1,2,4-oxadiazole, 3-(2-pyridyl)-5-(3,5-difluorophenyl)-1,2,4-oxadiazole, 3-(2-pyridyl)-5-(3-cyanophenyl)-1,2,4-oxadiazole, 3-(2-pyridyl)-5-(3,5-dimethoxyphenyl)-1,2,4-oxadiazol, 3-(2-pyridyl)-5-(2,3-dichlorophenyl)-1,2,4-oxadiazole, 3-(2-pyridyl)-5-(3-chloro-5-cyanophenyl)-1,2,4-oxadiazole, 3-(2-pyridyl)-5-(3-fluoro-5-cyanophenyl)-1,2,4-oxadiazole, 3-(2-pyridyl)-5-(3-chloro-5-fluorophenyl)-1,2,4-oxadiazole, 3-(5-chloropyrid-2-yl)-5-(3-cyanophenyl)-1,2,4-oxadiazole, 3-(5-fluoropyrid-2-yl)-5-(3-cyanophenyl)-1,2,4-oxadiazole, 3-(5-fluoropyrid-2-yl)-5-(3-cyano-5-fluorophenyl)-1,2,4-oxadiazole, 3-(3-fluoropyrid-2-yl)-5-(3-cyanophenyl)-1,2,4-oxadiazole, 3-(5-fluoropyrid-2-yl)-5-(3,5-dimethoxyphenyl)-1,2,4-oxadiazole, 3-(5-methoxypyrid-2-yl)-5-(3-cyanophenyl)-1,2,4-oxadiazole, 3-(2-quinolinyl)-5-(3-cyanophenyl)-1,2,4-oxadiazole, 3-(3-chloro-5-trifluoromethylpyrid-2-yl)-5-(3-cyanophenyl)-1,2,4-oxadiazole, 3-(2-pyridyl)-5-(5-chloro-2-methoxyphenyl)-1,2,4-oxadiazole, 3-(2-pyridyl)-5-(2-chloro-5-methylthiophenyl)-1,2,4-oxadiazole, 3-(2-pyridyl)-5-(2-bromo-5-methoxyphenyl)-1,2,4-oxadiazole, 3-(2-pyridyl)-5-(2,5,6-trifluorophenyl)-1,2,4-oxadiazole, 3-(2-pyridyl)-5-(3-nitrophenyl)-1,2,4-oxadiazole or 3-(2-pyridyl)-5-(3-bromophenyl)-1,2,4-oxadiazole.

According to one embodiment, the mGluRgIa may be an mGluR5 antagonist disclose3-in WO02068417A2, such as 3-(5-methyl-pyrid-2-yl)-5-(3-cyanophenyl)-1,2,4-oxadiazole, 3-(5-cyano-pyrid-2-yl)-5-(3-cyanophenyl)-1,2,4-oxadiazole, 3-(2-pyridyl)-5-(5-bromo-2-methoxyphenyl)-1,2,4-oxadiazoIe, 3-(2-pyridyl)-5-(5-bromo-2-fluorophenyl)-1,2,4-oxadiazole, 3-(2-pyridyl)-5-(5-cyano-2-fluorophenyl)-1,2,4-oxadiazole, 3-(2-pyridyl)-5-(5-bromopyrid-3-yl)-1,2,4-oxadiazole, 3-(2-pyridyl)-5-(5-chloro-pyrid-3-yl)-1,2,4-oxadiazole, 3-(5-cyanopyrid-2-yl)-5-(5-bromo-pyrid-3-yl)-1,2,4-oxadiazole, 3-(5-fluoropyrid-2-yl)-5-(5-bromo-pyrid-3-yl-1,2,4-oxadiazole, 3-(2-pyridyl)-5-(2-thiomethoxy-pyrid-3-yl) 1-1,2,4-oxadiazole, 3-(2-pyridyl)-5-(5-methylpyrid-3-yl)-1,2,4-oxadiazole, 3-(2-pyridyl)-5-(5-methoxypyrid-3-yl)-1,2,4-oxadiazole, 3-(2-pyridyl)-5-(3-cyano-5-methylphenyl)-1,2,4-oxadiazole, 3-(2-pyridyl)-5-(3-fluoro-5-bromophenyl)-1,2,4-oxadiazole, 3-(2-pyridyl)-5-(3-iodo-5-bromophenyl)-1,2,4-oxadiazole, 3-(5-fluoro-2-pyridyl)-5-(3-fluoro-5-bromophenyl)-1,2,4-oxadiazole, 3-(2-pyridyl)-5-(3-iodo-5-(methylphenylester)-1,2,4-oxadiazole, 3-(2-pyridyl)-5-(3-methoxy-5-(methoxycarbonyl)phenyl)-1,2,4-oxadiazole, 3-(2-pyridyl)-5-(3-bromo-5-cyanophenyl)-1,2,4-oxadiazole, 3-(2-pyridyl)-5-(5-cyano-3-iodophenyl)-1,2,4-oxadiazole, 3-(5-cyano-2-pyridyl)-5-(3-bromophenyl)-1,2,4-oxadiazole, 3-(5-cyano-2-pyridyl)-5-(3-cyano-5-fluorophenyl)-1,2,4-oxadiazole, 3-(5-cyano-2-pyridyl)-5-(3-bromo-5-fluorophenyl)-1,2,4-oxadiazole, 3-(2-pyridyl)-5-(5-cyano-2-methoxyphenyl)-1,2,4-oxadiazole, 3-(2-pyridyl)-5-(2-cyano-5-methoxyphenyl)-1,2,4-oxadiazole, 3-(2-pyridyl)-5-(5-cyano-pyrid-3-yl)-1,2,4-oxadiazole, 3-(2-pyridyl)-5-(3-cyano-5-(methoxycarbonyl)phenyl)-1,2,4-oxadiazole, 3-(2-pyridyl)-5-(5-phenyl-pyrid-3-yl)-1,2,4-oxadiazole, 3-(2-pyridyl)-5-(3-cyano-5-methoxyphenyl)-1,2,4-oxadiazole, 3-(2-pyridyl)-5-(3-cyano-5-hydroxyphenyl)-1,2,4-oxadiazoIe, 3-(2-pyridyl)-5-(3-cyano-5-propoxyphenyl)-1,2,4-oxadiazole, 2-(3-cyanophenyl)-4-(pyridin-2-yl)-1,3-thiazole, 2-(3-bromo-5-iodophenyl)-4-pyridin-2-yl)-1,3-oxazole, 2-(2-pyridyl)-5-(3-iodophenyl)-1,3,4-oxadiazole, 2-(2-pyridyl)-5-(3-cyanophenyl)-1,3,4-oxadiazole, 2-(2-pyridyl)-5-(3-cyanophenyl)-1,3,4-triazole, 3-(5-chloropyrid-2-yl)-5-(3-cyano-5-fluorophenyl)-1,2,4-oxadiazole, 3-(5-chloropyrid-2-yl)-5-(3-cyano-5-chlorophenyl)-1,2,4-oxadiazole, 3-(5-chloropyrid-2-yl)-5-(3-chloro-5-fluorophenyl)-1,2,4-oxadiazole, 3-(5-chloropyrid-2-yl)-5-(3-cyano-5-methoxyphenyl)-1,2,4-oxadiazole, 3-(5-fluoropyrid-2-yl)-5-(3-cyano-5-chlorophenyl)-1,2,4-oxadiazole, 3-(5-fluoropyrid-2-yl)-5-(3-fluoro-5-chlorophenyl)-1,2,4-oxadiazole, 3-(5-fluoropyrid-2-yl)-5-(3-cyano-5-methoxyphenyl)-1,2,4-oxadiazole, 3-(5-cyanopyrid-2-yl)-5-(3-cyano-5-chlorophenyl)-1,2,4-oxadiazole, 3-(5-cyanopyrid-2-yl)-5-(3-fluoro-5-chlorophenyl)-1,2,4-oxadiazole, 3-(5-cyanopyrid-2-yl)-5-(3-cyano-5-methoxyphenyl)-1,2,4-oxadiazole, 3-(5-fluoropyrid-2-yl)-5-(3,5-di-cyanophenyl)-1,2,4-oxadiazole, 3-(3-(4-dimethylaminobutoxy)-pyrid-2-yl)-5-(3-cyano-5-fluorophenyl)-1,2,4-oxadiazole, 3-(3-(5-dimethylaminopentyloxy)-pyrid-2-yl)-5-(3-cyano-5-fluorophenyl)-1,2,4-oxadiazole or [3-(3-(6-dimethylaminohexyloxy)-pyrid-2-yl)-5-(3-cyano-5-fluorophenyl)-1,2,4-oxadiazole.

According to one embodiment, the mGluRgIa may be an mGluR5 antagonist disclosed in WO2005066155A1, such as 3-fluoro-5-[5-(5-fluoropyridin-2-yl)-2H-tetrazol-2-yl]benzonitrile, 6-[2-(3-cyano-5-fluorophenyl)-2H-tetrazol-5-yl]nicotinonitrile, 3-[5-(5-chloropyridin-2-yl)-2H-tetrazol-2-yl]-5-fluorobenzonitrile, 3-[5-(5-fluoro-pyridin-2-yl)-tetrazol-2-yl]-5-methoxymethyl-benzonitrile, 3-fluoro-5-[2-(5-fluoropyridin-2-yl)-2H-tetrazol-5-yl]benzonitrile, 6-[5-(3-cyano-5-fluorophenyl)-2H-tetrazol-2-yl]nicotinonitrile, 3-[2-(5-chloropyridin-2-yl)-2H-tetrazol-5-yl]-5-fluorobenzonitrile, 3-[5-(5-fluoropyridin-2-yl)-2H-tetrazol-2-yl]-5-(methoxymethyl)benzonitrile, 5-fluoro-2-[2-(3-fluoro-5-methoxyphenyl)-2H-tetrazol-5-yl]pyridine, 3-[5-(5-fluoro-pyridin-2-yl)-2H-tetrazol-2-yl]-5-methoxybenzonitrile, 3-[5-(5-fluoropyridin-2-yl)-2H-tetrazol-2-yl]-5-(trifluoromethoxy)benzonitrile, 3-(difluoromethoxy)-5-[5-(5-fluoropyridin-2-yl)-2H-tetrazol-2-yl]benzonitrile, 3-[5-(5-fluoropyridin-2-yl)-2H-tetrazol-2-yl]-5-(2-methoxyethoxy)benzonitrile, 3-(ethylamino)-5-[5-(5-fluoropyridin-2-yl)-2H -tetrazol-2-yl]benzonitrile, 3-amino-5-[5-(5-fluoropyridin-2-yl)-2H-tetrazol-2-yl]benzonitrile or 3-[5-(5-fluoropyridin-2-yl)-2H-tetrazol-2-yl]-5-iodobenzonitrile.

According to one embodiment, the mGluRgIa may be an mGluR5 antagonist disclosed in WO2004000316A1, such as 3-[3-(5-fluoropyridin-2-yl)-1,2,4-oxadiazol-5-yl]-5-(methoxymethyl)benzonitrile or 3-fluoro-5-[3-(5-fluoropyridin-2-yl)-1,2,4-oxadiazol-5-yl]benzonitrile.

According to one embodiment, the mGluRgIa may be an mGluR5 antagonist disclosed in WO2005080379A1, such as 3- (3-chlorophenyl)-5-{[(4-methyl-5-pyridin-3-yl-4H-1,2,4-triazol-3-yl)thio]methyl}-1,3,4-oxadiazol-2(3H)-one, 2-(3-chlorophenyl)-5-{1-[methyl(4-methyl-5-pyridin-4-yl-4H-1,2,4-triazol-3-yl)amino]ethyl}-2,4-dihydro-3H-1,2,4-triazol-3-one, 4-(5-{1-[1- (3-chlorophenyl)-1H-pyrazol-4-yl]ethoxy}-4-methyl-4H-1,2,4-triazol-3-yl) pyridine, 4-(5-{1-[2-(3-chlorophenyl)-2H-1,2,3-triazol-4-yl]ethoxy}-4-methyl-4H-1,2,4-triazol-3-yl)pyridine, 4-[5-({1-[2-(3-chlorophenyl)-2H-1,2,3-triazol-4-yl]ethyl}thio)-4-cyclopropyl-4H-1,2,4-triazol-3-yl]pyridine, 4-{5-[1-(3-chloro-phenyl)-1H-[1,2,4]triazol-3-ylmethylsulfanyl]-4-cyclopropyl-4H[1,2,4]triazol-3-yl}-pyridine, 4-{5-[1-(3-chloro-phenyl)-1H-[1,2,4]triazol-3-ylmethoxy]-4-cyclopropyl-4H[1,2,4]triazol-3-yl}-pyridine, 4-{5-[1-(3-chloro-phenyl)-1H-[1,2,3]triazol-4-ylmethylsulfanyl]-4-methyl-4H[1,2,4]triazol-3-yl}-pyridine, 4-{5-[1-(3-chloro-phenyl)-1H-[1,2,3]triazol-4-ylmethylsulfanyl]-4-cyclopropyl-4H[1,2,4]triazol-3-yl}-pyridine, 4-{5-[1-(3-chloro-phenyl)-1H-[1,2,3]triazol-4-ylmethoxy]-4-cyclopropyl-4H-[1,2,4]triazol-3-yl}-pyridine, or 4-(5-{(1R)-[2-(3-chlorophenyl)-2H-1,2,3-triazol-4-yl]ethoxy}-4-methyl-4H-1,2,4-triazol-3-yl)pyridine.

According to one embodiment, the mGluRgIa may be an mGluR5 antagonist disclosed in WO2005080397A2, such as 7-[5-(5-Chloro-2-fluorophenyl)-1,2,4-oxadiazol-3-yl]-3-(2-thienyl)-6,7-dihydro-5H[1,2,4]triazolo[3,4-b][1,3]thiazine, 9-{[5-(3-chlorophenyl)-1,2,4-oxadiazol-3- yl]methyl}-3-pyridin-4- y1-6,7,8,9-tetrahydro-5H-[1,2,4]triazolo[4,3-a][1,3]diazepine, 9-{1-[5-(3-chlorophenyl)-1,2,4-oxadiazol-3-yl]ethyl}-3-pyridin-4-yl-6,7,8,9-tetrahydro-5H-[1,2,4]triazolo[4,3-a][1,3]diazepine, 7-{[5-(3-chlorophenyl)-1,2,4-oxadiazol-3-yl]methyl}-3-pyridin-4-y1-6,7-dihydro-5Hpyrrolo[2,1-c][1,2,4]triazole, 9-{[5- (3-chlorophenyl)-1,2,4-oxadiazol-3- yl]methyl}-3-(trifluoromethyl)-6,7,8,9-tetrahydro-5H-[1,2,4]triazolo[4,3-a][1,3]diazepine, 8-[3-(3-chloro-phenyl)-[1,2,4]oxadiazol-5-yl]-3-(4-methoxy-phenyl)-5,6,7,8-tetrahydro-[1,2,4]triazolo[4,3-a]pyrazine, 8-[3-(3-chloro-phenyl)-[1,2,4]oxadiazol-5-yl]-3-(4-methoxy-phenyl)-7-methyl-5,6,7,8-tetrahydro-[1,2,4 ]triazolo[4,3-a]pyrazine, 9-{[5-(3-chlorophenyl)is oxazol-3-yl]methyl}-3-(3,5-difluorophenyl)-6,7,8,9-tetrahydro-5H-[1,2,4]triazolo[4,3-a][1,3]diazepine, 9-{[5-(3-chlorophenyl)isoxazol-3-yl]methyl}-3-(4-methoxyphenyl)-6,7,8,9-tetrahydro-5H-[1,2,4]triazolo[4,3-a][1,3]diazepine, 9-{[5-(3-chlorophenyl)is oxazol-3-yl]methyl}-3-pyridin-4-yl-6,7,8,9-tetrahydro-5H[1,2,4]triazolo [4,3-a][1,3]diazepine, 9-{[5- (5-chloro-2-fluorophenyl)-1,2,4-oxadiazol-3-yl]methyl}-3-pyridin-4-yl-6,7,8,9-tetrahydro-5H-[1,2,4]triazolo[4,3-a][1,3]diazepine, 9-{[5-(3-chlorophenyl)-1,2,4-oxadiazol-3-yl]methyl}-3-(3,5-difluorophenyl)-6,7,8,9-tetrahydro-5H-[1,2,4]triazolo[4,3-a][1,3]diazepine or 9-{[5-(3-chlorophenyl)-1,2,4-oxadiazol-3-yl]methyl}-3-(4-methoxyphenyl)-6,7,8,9-tetrahydro-5H-[1,2,4]triazolo[4,3-a][1,3]diazepine.

According to one embodiment, the mGluRgIa may be an mGluR5 antagonist disclosed in WO2007040982A1, such as 3-{5-{(1R)-1-[5-{3-chlorophenyl)isoxazol-3-yl]ethoxy}-4-methyl-4H-1,2,4-triazol-3-yl) pyridine, 4-(5-{(rac)-1-[5- (3-chlorophenyl)isoxazol-3-yl]ethoxy}-4-methyl-4H-1,2,4-triazol-3-yl) pyridine, 4-(5-{(1R)-1-[5-(3-chlorophenyl)isoxazol-3-yl]ethoxy}-4-methyl-4H-1,2,4-triazol-3-yl) pyridine or 4-(5-{(1S)-1-[5-(3-chlorophenyl)isoxazol-3-yl]ethoxy}-4-methyl-4H-1,2,4-triazol-3-yl) pyridine.

According to one embodiment, the mGluRgIa may be the mGluR5 antagonist 4-(5-{(1R)-1-[5-(3-chlorophenyl)isoxazol-3-yl]ethoxy}-4-methyl-4H-1,2,4-triazol-3-yl) pyridine disclosed in WO2010019100A1.

According to one embodiment, the mGluRgIa may be predominantly peripherally acting, i.e. have a limited ability of passing the blood-brain barrier. After IV or oral administration, the ratio between the concentration of the mGluRgIa in the brain and in the blood-plasma may be less than 0.3, such as less than 0.1 or 0.01. Advantages of an mGluRgIa with limited ability of passing the blood-brain barrier in applications according to the invention include a minimized risk of CNS related undesired side effects.

According to one embodiment, the mGluRgIa may be a predominantly peripherally acting mGluR5 antagonist disclosed in WO2007130820A2, such as 5- (5-{(R)-2-[5- (3-chloro-phenyl)-isoxazol-3-yl]-pyrrolidin-1-yl}-4-methyl-4H-[1,2,4]triazol-3-yl)-1-methyl-1H-pyridin -2-one, 4-(5-{(R)-2-[5-(3-chloro-phenyl)-isoxazol-3-yl]-pyrrolidin-1-yl}-4-methyl-4H-[1,2,4]triazol-3-yl)-pyridazine, 4-(5-{(R)-2-[2-(3-chloro-phenyl)-2H-tetrazol-5-yl]-pyrrolidin-1-yl}-4-methyl-4H[1,2,4]triazol-3-yl)-1-methyl-1H-pyridin-2-one or 1-methyl-4-{4-methyl-5-[(R)-2- (5-m-tolylisoxazol-3-yl)pyrrolidin-1-yl]-4H-[1,2,4]triazol-3-yl}-1H-pyridin-2-one.

According to one embodiment, the mGluRgIa may be a predominantly peripherally acting mGluR5 antagonist disclosed in WO2007130821A2, such as 3-(5-{1-[5-(2-methoxy-pyridin-4-yl)-4-methyl-4H[1,2,4]triazol-3-yl]-piperidin-2-yl}-tetrazol-2-yl)-bcnzonitrile or 3-(5-{(S)-2-[2-(3-chloro-phenyl)-2H-tetrazol-5-yl]-piperidin-1-yl}-4-methyl-4H-[1,2,4]triazol-3-yl)-pyridine.

According to one embodiment, the mGluRgIa may be a predominantly peripherally acting mGluR5 antagonist disclosed in WO2007130822A2, such as 4-(5-{(R)-2-(2-(3-chloro-phenyl)-2H-tetrazol-5-yl]-pyrrolidin-1-yl}-4-methyl-4H-[1,2,4]triazol-3-yl)-2-methyl-pyridine or 3-(5-{(R)-1-[5-(2-methoxy-pyridin-4-yl)-4-methyl-4H-[1,2,4]triazol-3-yl]-pyrrolidin-2-y}l-tetrazol-2-yl)-benzonitrile.

According to one embodiment, the mGluRgIa may be a predominantly peripherally acting mGluR5 antagonist disclosed in WO2007130823A2, such as 3-(3-{(R)-1-[4-methyl-5-(2-methyl-pyridin-4-yl)-4H-[1,2,4]triazol-3-yl]-pyrrolidin-2-yl}-isoxazol-5-yl)-benzonitrile or 3-{3-[(R)-1-(4-methyl-5-pyridin-3-yl-4H-[1,2,4]triazol-3-yl)-pyrrolidin-2-yl]-isoxazol-5-yl}-benzonitrile.

According to one embodiment, the mGluRgIa may be a predominantly peripherally acting mGluR5 antagonist disclosed in WO2007130824A2, such as 3-pyridin-3-yl-8-(2-m-tolyl-2H-tetrazol-5-ylmethyl)-5,6,7,8-tetrahydro[1,2,4]triazolo[4,3-a]pyrimidine, 8-{[5-(3-chlorophenyl)-1,2,4-oxadiazol-3-yl]methyl-3-pyridin-3-yl-5,6,7,8-tetrahydro[1,2,4]triazolo[4,3-a]pyrimidine, 8-[5-(3-chloro-phenyl)-[1,2,4]oxadiazol-3-ylmethyl]-3-pyrimidin-5-yl-5,6,7,8-tetrahydro-[1,2,4]triazolo[4,3-a]pyrimidine or 5-(8-{1-[5-(3-chloro-phenyl)-isoxazol-3-yl]-ethyl}-5,6,7,8-tetrahydro-[1,2,4]triazolo[4,3-a]pyrimidin-3-yl)-2H-pyridazin-3-one.

According to one embodiment, the mGluRgIa may be a predominantly peripherally acting mGluR5 antagonist disclosed in WO2008041075A1, such as 4-(5-{(1R)-1-[5- (3-Chlorophenyl)isoxazol-3-yl]ethoxy}-4-methyl-4H-1,2,4-triazol-3-yl)pyridin-2(1H)-one.

According to one embodiment, the mGluRgIa may be a predominantly peripherally acting mGluR5 antagonist disclosed in WO2009054787A1, such as 4-[5-[(2R)-2-[5-(3-chlorophenyl)1,2-oxazol-3-yl]pyrrolidin-1-yl]-4-methyl-1,2,4-triazol-3-yl]benzamide or 4-[5-[(2R)-2-[5-(3-chlorophenyl)1,2-oxazol-3-yl]pyrrolidin-1-yl]-4- methyl-1,2,4-triazol-3-yl]-N-methylbenzamide.

According to one embodiment, the mGluRgIa may be a compound having dual mGluR1/mGluR5 antagonist properties disclosed in WO9926927A2, such as 3-(1-adamantanemethoxy)-2-chloroquinoxaline, 2-(1-adamantanemethoxy)-3-methylquinoxaline, 3-(1-adamantanemethoxy)-2-fluoroquinoxaline, 2-(1-adamantanemethoxy)-3-trifluoromethylquinoxaline, S-{2-(4-phenylthiazolyl)1-1-adamantanecarboxamide, N-{2-(5-methyl-4-phenylthiazolyl)]-1-adamantanecarboxamide, 1-(1-adamantyl)-2-(benzothiazol-2-ylsulfanyl)ethanone, N-(1-adamantyl)-2-chloroquinoxaline-3-carboxamide, N-(1-adamantyl)-3-methylquinoxaline-2-carboxamide, N-(1-adamantyl)-1-oxyquinoxaline-3-carboxamide, 4-chlorophenyl 3-coumarincarboxylate, 2-{1-adamantanemethylsulfanyl)quinoxaline, 3-(1-adamantanemethoxy)-2-chloropyrazine, 1-(1-adamamyl)-2-(4,6-dimethylpyrimidin-2-ylsulfanyl)ethanone, 1-(1-adamantyl)-2-(2-anisylsulfanyl)ethanone, 3-(1-adamantanemethoxy)-1H-quinoxalin-2-one, 1-(1-adamantyl)-2-(3-anisylsulfanyl)ethanone, 1-(1-adamantyl)-2-(4-anisylsulfanyl)ethanone, 1-(1-adamantyl)-2-(4-chlorophenylsulfanyl)ethanone, 1-(1-adamantyl)-2-(2-naphthylsulfanyl)ethanone, N-(2-{6-(1-Piperidinyl)pyrazinyl])-ladamantanecarboxamide, N-(2-{6-(1-Piperidinyl)pyrazinyll)adamantan-1-ylmethylcarboxamide, 1-(1-adamamyl)-2-(1-naphthylsulfanyl)ethanone, 1-(1-adamamyl)-2-(S-quinolylsulfanyl)ethanone hydrochloride, 1-(1-Adamantyl)-2-(4-trifluoromethoxyphenoxy)ethanone, 2-(1-adamantanemethoxy)quinoxaline, N(trans-4-methylcyclohexyl)-2-quinoxalinecarboxamide, N-(cis-4-methylcyclohexyl)-2-quinoxalinecarboxamide, N-(trans-4-methylcyclohexyl)-2-quinolinecarboxamide, N-(trans-4-methylcyclohexyl)-3-quinolinecarboxamide, N-(trans-4-methylcyclohexyl)-6-quinolinecarboxamide, 2-(1-adamantanemethylsulfinyl)-benzothiazole, N-(4-phenyl butyl)-2-quinoxalinecarboxamide, 1-(1-adamantyl)-2-(4,6-dimethylpyrimidin-2-ylsulfanyl)ethanol, 1-(1-adamantyl)-2-(3-chloroquinoxal-2-yl)ethanone, 2-(1-adamantanemethylsulfanyl)-3-methylquinoxaline, N-(1-adamantyl)-2-anisamide, N-(1-adamamanemethyl)-2-anisamide, 1-(1-adamantyl)-2-(4-chlorophenylsulfanyl)ethanone, 2-(1-adamantanemethylsulfonyl)-3-methylquinoxaline, 1-(1- adamantyl)-2-(4-tluorophenylsulfanyl)ethanone, 1-(1-adamantyl)-2-(3-fluorophenylsulfanyl)ethanone, 1-(1-adamantyl)-2-(2-methoxyphenoxy)erhanone, 1-(4-anisylsulfanyl)butan-2-one, 1-(1-adamantyl)-2-(4-anisidinyl)ethanone hydrochloride, 3,3-dimethyl-1-(4-anisylsulfanyl)butan-2-one, 1-(4-biphenyl)-2-(4-anisylsulfanyl)ethanone, 1-(1-adamantyl)-2-(2-trifluoromethoxyphenylsulfanyl)ethanone, 1-(1-adamantyl)-2-(3-methylquinoxal-2-ylsulfanyl)ethanone, 1-(1-adamantyl)-2-(2-anisidinyl)ethanone hydrochloride, 1-(1-adamantyl)-2-(4-tritluoromerhoxyphenylamino)ethanone hydrochloride, 1-(1-adamantyl)-2-(N-methyl-4-anisidinyl)ethanone hydrochloride, N-(1-adamantyl)-7-fluoromethylquinoline-3-carboxamide, N-(1-adamantyl)-2-(1-piperizinyl)quinoxaline-3-carboxamide, N-(1-adamantyl)-2-(2-aminoethylamino)quinoxaline-3-carboxamide, methyl N-(3-quinolyl)-3-carboxyadamantane-1-carboxamide, 1-(1-adamantyl)-2-[(R)-1-(1-naphthyl)ethan-1-ylaminolethanone, N-(1-adamantyl)-2-methoxyquinoxaline-3-carboxamide, ethyl N-(1-adamantyl)-2-(3-propanoylamino)quinoxaline-3-carboxamide, N-(4-chlorophenyl)-2,3-dimethylquinoxaline-6-carboxamide, N-(1-adamantyl)-6,7-dimethylquinoxaline-2-carboxamide, N—(S)-1-tetralinyl)-2-quinoxalinecarboxamide, N-(4-chlorophenethyl)-2-quinoxalinecarboxamide, N-(6-quinolyl)-2-quinoxalinecarboxamide, N-(1-tetralinmethyl)-2-quinoxalinecarboxamide, N-(1-indanmethyl)-2-quinoxalinecarboxamide or N-(4,4-dimethylcyclohexyl)-2-quinoxalinecarboxamide.

According to one embodiment, the mGluRgIa may be a compound being a more potent mGluR1 antagonist than an mGluR5 antagonist disclosed in WO2004069813A1, such as N-(trans-4-methylcyclohexyl)-5,6,7,8-tetrahydroquinoxaline-2-carboxamide, N-(4,4-dimethylcyclohexyl)-5,6,7,8-tetrahydroquinoxaline-2-carboxamide.

Any of the herein mentioned compounds having mGluRgIa activity may be used in applications according to the invention as a mixture of diastereomers, a pure diastereomer with mGluRgIa activity, a racemic mixture, a scalemic mixture or a pure enantiomer with mGluRgIa activity.

According to one embodiment, there is provided a pharmaceutical composition comprising a compound having mGluRgIa activity and at least one pharmaceutically acceptable carrier or excipient. Such a pharmaceutical composition may further comprise one or more different therapeutic agents. Preferably, the one or more different therapeutic agents are selected from a group with a mechanism of action that differs from the mechanism of action of a compound being an mGluRgIa. An advantageous synergistic effect between the therapeutic agents may then occur, allowing a more effective combat or prevention of e.g. undesired union of tissue, in particular intra abdominal adhesions, than if only one of the therapeutic agents is used. For example, an mGluRgIa, such as a suitable mGluRgIa compound mentioned herein, may be combined with one or more of the group consisting of anticoagulants, fibrinolytics, thromboxane A2 receptor blockers, anti-inflammatories, antihistamines, growth factor inhibitors, epidermal growth factor, ACE inhibitors, angiotensin II receptor blockers, matrix metalloproteinases, immunosuppressives, octreotide, systemic and locally administered antibiotics, taurolidine, phospholipids, collagen inhibitors, medroxyprogesterone and leuprolide acetate, hypoestrogenic environment inducers, aromatase inhibitors, phosphodiesterase 5 inhibitors, methylene blue, catalase, vitamin E, opioids, local anesthetics, sphingosine kinase 1 via adenovirus, neurokinin-1 receptor antagonists and ionotropic glutamate receptor antagonists such as an NMDA antagonist.

According to one embodiment, an mGluRgIa compound, or a pharmaceutical composition comprising an mGluRgIa compound, may be administered in combination with the use of a physical barrier as known in the art, such as a solid sheet of carboxymethylcellulose and hyaluronic acid, or an oxidized sheet of regenerated cellulose, for the prevention of abnormal union of tissue, such as intra abdominal adhesions occurring after surgery.

According to one embodiment, there is provided a method for the treatment or prevention of undesired union of tissue, such as intra abdominal adhesions, wherein an effective amount of an mGluRgIa compound, or an effective amount of a pharmaceutical composition as disclosed herein, is administered to a subject in need of such treatment or prevention.

A pharmaceutical composition according to embodiments disclosed herein may be administered through different routes such as, but not limited to, intravenously, intraperitonealy, intramuscularly, intranasaleously, subcutaneously, cutaneously, sub-lingually, rectally, orally, through inhalation or insufflations or sprayed or smeared locally on organs and wounds during surgery.

An mGluRgIa compound, or a pharmaceutically acceptable salt or solvate thereof, may be formulated into conventional pharmaceutical compositions, e.g. medicaments for treatment or prevention of undesired union of tissue, such as intra abdominal adhesions. The pharmaceutical composition may comprise an mGluRgIa compound in association with a pharmaceutically acceptable carrier or excipient.

A pharmaceutical composition, as described herein, may further comprise pharmaceutically diluents, stabilizers and the like.

The pharmaceutically acceptable carriers may be either solid or liquid. Solid form preparations include, but are not limited to, powders, tablets, dispersible granules, capsules, cachets, and suppositories.

A solid carrier may be one or more substances, which may also act as diluents, flavoring agents, solubilizers, lubricants, suspending agents, binders, or tablet disintegrating agents. A solid carrier may also be an encapsulating material. In powders, the carrier is a finely divided solid, which is in a mixture with a finely divided mGluRgIa. In tablets, the mGluRgIa is mixed with the carrier having the necessary binding properties in suitable proportions and compacted in the shape and size desired.

For preparing suppository compositions, a low-melting wax such as a mixture of fatty acid glycerides and cocoa butter is first melted and the mGluRgIa is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the mGluRgIa with encapsulating material as a carrier providing a capsule in which the mGluRgIa (with or without other carriers) is surrounded by a carrier which is thus in association with it. Similarly, cachets are included.

Tablets, powders, cachets, and capsules may be used as solid dosage forms suitable for oral administration.

Liquid form compositions include solutions, suspensions, and emulsions. For example, sterile water or water propylene glycol solutions of the mGluRgIa may be liquid preparations suitable for parenteral administration. Liquid compositions may also be formulated in solution in aqueous polyethylene glycol solution.

Aqueous solutions for oral administration may be prepared by dissolving the mGluRgIa in water and adding suitable colorants, flavoring agents, stabilizers, and thickening agents as desired. Aqueous suspensions for oral use may be made by dispersing the finely divided mGluRgIa in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, or from about 0.10% w to 50% w, of an mGluRgIa, all percentages by weight being based on the total weight of the composition.

A therapeutically effective amount for the practice of the present invention may be determined by one of ordinary skill in the art using known criteria including the age, weight and response of the individual patient, and interpreted within the context of the disease which is being treated or which is being prevented.

‘Pharmaceutically acceptable’ means an excipient that, at the dosage and concen- trations employed, does not cause any unwanted effects in the patients to whom it is administered. Such pharmaceutically acceptable excipients are well-known in the art.

A pharmaceutical composition according embodiments herein may be administered to a patient in a pharmaceutically effective dose. By ‘pharmaceutically effective dose’ is meant a dose that is sufficient to produce the desired effects in relation to the condition for which it is administered. The exact dose may be dependent on the activity of the mGluRgIa, manner of administration, nature and severity of the disorder and/or disease and the general conditions, such as age and body weight of the patient.

According to an embodiment, an mGluRgIa compound, or a pharmaceutical composition comprising an mGluRgIa, may be useful in the treatment or prevention of abnormal union of tissue. Examples of abnormal union of tissue include intra abdominal adhesions in general, intra abdominal adhesions occurring after surgery, such as surgery wherein the peritoneum is pierced, and adhesions between an organ or tissue and an implant, such as a breast implant or an artificial bone or articular.

According to an embodiment, an mGluRgIa compound, or a pharmaceutical composition comprising an mGluRgIa, may be useful in the prevention or reduction of intra abdominal adhesions commonly occurring after abdominal surgery. A therapeutically effective amount of the mGluRgIa compound, or a pharmaceutical composition comprising an mGluRgIa compound, may be administered to the patient just before, during, or just after surgery, to prevent or reduce post-operative intra abdominal adhesions. The mGluRgIa compound, or the pharmaceutical composition comprising such, may be administered e.g. 0 to 5 hours before surgery, such as by e.g. injection into the abdominal cavity. The mGluRgIa compound, or the pharmaceutical composition comprising such, may also be administered during surgery, such as just before closing of the surgical cut, by e.g. spraying or soaking the exposed abdominal organs and/or tissue. The mGluRgIa compound, or the pharmaceutical composition comprising such, may be administered e.g. 0 to 2 hours after surgery, preferably 0 to 0.5 hours after surgery, such as by e.g. injection into the abdominal cavity.

Further, anmGluRgla compound, or a pharmaceutical composition comprising an mGluRgIa, may be used in a method for treating or preventing abnormal union of tissue. Such a method may include the step of administering an effective amount of an mGluRgIa compound, or an effective amount of a pharmaceutical composition as disclosed herein, to a subject that that is in need of such treatment or prophylaxis.

In the context of the present specification, the term ‘therapy’ and ‘treatment’ includes prevention or prophylaxis, unless there are specific indications to the contrary. The terms ‘therapeutic’ and ‘therapeutically’ should be construed accordingly.

According to one embodiment, treatment does also encompass pre-treatment, i.e. prophylactic treatment.

When used herein, ‘prevent/preventing’ should not be construed to mean that a condition and/or a disease never might occur again after use of a compound or pharmaceutical composition according to embodiments disclosed herein to achieve prevention. Further, the term should neither be construed to mean that abnormal union of tissue not might occur, at least to some extent, after such use to prevent abnormal union of tissue. Rather, ‘prevent/preventing’ is intended to mean that abnormal union of tissue to be prevented, if occurring despite such use, will be less severe than without such use.

According to one embodiment, a pharmaceutical composition according to embodiments herein may be administered alone or in combination with other therapeutic agents. These agents may be incorporated as part of the same pharmaceutical composition or may be administered separately. It is well known in the art that a combination of mechanistically unrelated therapeutic agents in the same medicament may have beneficial effects in the treatment of conditions or diseases characterized by e.g. abnormal union of tissue.

When an mGluRgIa according to embodiments disclosed herein is combined with at least another therapeutic agent, such as a therapeutic agent belonging to one of the groups consisting of anticoagulants, fibrinolytics, thromboxane A2 receptor blockers, anti-inflammatories, antihistamines, growth factor inhibitors, epidermal growth factor, ACE inhibitors, angiotensin II receptor blockers, matrix metalloproteinases, immunosuppressives, octreotide, systemic and locally administered antibiotics, taurolidine, phospholipids, collagen inhibitors, medroxyprogesterone and leuprolide acetate, hypoestrogenic environment inducers, aromatase inhibitors, phosphodiesterase 5 inhibitors, methylene blue, catalase, vitamin E, opioids, local anesthetics, sphingosine kinase 1 via adenovirus, neurokinin-1 receptor antagonists and ionotropic glutamate receptor antagonists such as an NMDA antagonist, in a pharmaceutical composition, such as a medicament, a therapeutically effective dose of the pharmaceutical composition may comprise 1 to 10 times less than the respective established therapeutically effective dose of a component, i.e. an mGluRgIa or the therapeutic agent, when administered alone for prevention or treatment of the same disease or condition.

Accordingly, by combining an mGluRgIa according to embodiments disclosed herein with another therapeutic agent, it may be possible to achieve synergistic effects compared to if only an mGluRgIa, or the other therapeutic agent, were administrated alone.

According to one embodiment, an mGluRgIa, or a pharmaceutical composition comprising an mGluRgIa, may be used for the prevention of abnormal union of tissue in humans or animals, such as dogs, cats, horses, cows or other mammals, in particular domestic animals. Animals may be treated for the same diseases and conditions as humans may be treated for.

The pharmacological properties of themGluRglacompounds, described herein as useful for the treatment of abnormal union of tissue, can be analyzed using standard assays for functional activity. Examples of glutamate receptor assays are well known in the art as described in for example Aramori et at., Neuron 8:757 (1992), Tanabe et al., Neuron 8:169 (1992), Miller et al., J. Neuroscience 15: 6103 (1995), Balazs, et at., J. Neurochemistly 69:151 (1997). The methodology described in these publications is incorporated herein by reference. The e.g. potency of the mGluRgIa compounds of the invention can e.g. be studied by means of various well known assays, such as e.g.FLIPR,that measures the mobilization of intracellular calcium in cells expressing mGluR1 or mGluR5, or recombinant versions thereof.

Additional properties of an mGluRgIa, which are of importance for the skilled person to be able to assess the suitability of a certain mGluRgIa for use in thetreatment of abnormal union of tissue, include potency, affinity, onset of action and biological half-life. Potency is herein meant to be understood as a measure, e.g. an IC50 value, of the amount, e.g. concentration, of an mGluRgIa to elicit half inhibition of the maximum biological response of an agonist, e.g. glutamate, when acting at mGluR group I receptors. Onset of action is herein meant to be understood as the duration of time it takes for the effect of an mGluRgIa to come to prominence upon administration. The biological half-life of an mGluRgIa is herein meant to be understood as the time it takes for the mGluRgIa to lose half of its pharmacologic activity, measured as e.g. the inhibition of an agonists ability to activate phospholipase C via mGluR group I receptors or any other mGluR group I response upon action of an agonist, at the target site. The target site may be the tissue or organ from which connective tissue is formed to generate undesired union of tissue, or remote tissue or organs expressing mGluR group I receptors which is communicating with the tissue or organ from which connective tissue is formed to generate undesired union of tissue via e.g. nerve signaling or the immune system. It is well known to the one skilled in the art how to measure and/or estimate potency, affinity, onset of action and biological half-life of a particular mGluRgIa. Suitable experiments, including in-vitro, in-vivo and in-silico experiments, and methodologies for such measurements/estimations are well known to the skilled person. After having collected such measures/estimates of a particular mGluRgIa by standard experiments and methods, the skilled person can easily, based on these measures, make an assessment of the suitability of that particular mGluRgIa for treatment of abnormal union of tissue. Hence, the potency, affinity, onset of action and biological half-life of a particular mGluRgIa are all measures from which an overall ‘drug suitability score’ may be determined for the mGluRgIa in question. Such drug suitability scores may easily be determined by the skilled person for several different mGluRgIa compounds, whereby these may be ranked in order of suitability for treatment of abnormal union of tissue. The drug suitability score is positively correlated to the potency (high potency is reflected by a low IC50), the affinity, the biological half-life of a compound, such as an mGluRgIa. A compound, such as an mGluRgIa, with a high drug suitability score, has a higher probability of being more suited for a particular indication, such as in the treatment of abnormal union of tissue, than another compound with a lower drug suitability score.

According to one embodiment, the drug suitability score of an mGluRgIa of the invention may be comparable to or higher than the drug suitability score of MPEP, or any other drug, investigative compound or tool compound, such as compounds mentioned herein, acting as an antagonist of mGluR1 or mGluR5, or both of mGluR1 and mGluR5. Such drug suitability score may be calculated or estimated on the basis of one or several of potency, affinity, onset of action and biological half-life. Potency or affinity may be measured or estimated as potency or affinity of an mGluRgIa at mGluR group I receptors, mGluR5 receptors or mGluR1 receptors. Potency or affinity of an mGluRgIa at mGluR group I receptors may be estimated as the potency or affinity, respectively, at mGluR1 or mGluR5 receptors. An mGluRgIa with a drug suitability score equal to or higher than a comparative mGluRgIa compound may be as least as effective for the treatment of abnormal union of tissue as the comparative mGluRgIa compound is for the treatment of a decease or condition which the latter has been developed for, is effective for, or is claimed to be effective for.

According to one embodiment, mGluRgIa compounds of the invention may have an estimated drug suitability score which is greater than or equal to the drug suitability score for a comparative compound having a pharmaceutical effect other than affecting abnormal union of tissue due to antagonism of either or both of mGluR1 and mGluR5. Such mGluRgIa compounds are expected to be moderately to highly effective for the treatment of abnormal union of tissue, depending on the magnitude of the drug suitability score.

According to one embodiment, mGluRgIa compounds of the invention may have a pharmaceutical effect other than affecting abnormal union of tissue due to antagonism of either or both of mGluR1 and mGluR5. Such mGluRgIa compounds are expected to be moderately to highly effective for the treatment of abnormal union of tissue, depending on the magnitude of the pharmaceutical effect which is different than affecting abnormal union of tissue.

According to one embodiment, the IC50 value of anmGluRgla of the invention, e.g. as measured by a typical FLIPR assay employing cells expressing either mGluR1 or mGluR5, is typically less than 5000 nM, such as e.g. less than 1000, 500 or 100 nM. Compounds with such IC50 values may be considered as at least moderately effective mGluRgIa for the prevention of abnormal union of tissue.

According to one embodiment, the potency, estimated by e.g. the IC50 in-vitro at mGluR5 in a suitable known assay, of the mGluRgIa of the invention, may be at least comparable to the potency of 2-methyl-6-(phenylethynyl)-pyridine (MPEP). An mGluRgIa antagonist which is at least as potent at the mGluR5 receptor as MPEP is expected to be highly effective in the prevention of abnormal union of tissue. Support for this may be found, for example, by an example described herein.

### EXAMPLES

### Example 1

Reduction in the occurrence of post-operative intra abdominal adhesions in rat by treatment with the mGluRgIa compound 2-methyl-6-(phenylethynyl)-pyridine (MPEP) at a dose of 12.5 mg/kg

Procedure:

12 male Sprague-Dawleys rats (Charles River Laboratories International, Sutzfeld, Germany), weighing between 250 to 300 g at the initiation of the procedure, were kept at a number of two individuals in one cage of type H3, lined with wood chips and housed in standardized conditions, at a constant room temperature (22° C.) with 12-hour light and dark cycles. All animals were provided free access to standard rodent chow, tap water, nesting material and libitum. All surgical procedures were performed under aseptic conditions in a dedicated surgical animal operating room. The animals were anesthetized using continuous inhalation isoflurane 1.5% to 4% in oxygen, numbered and randomly assigned to two groups of 6 individuals each: one treatment group and one vehicle group. After shaving and disinfection of the abdomen, a 50 mm long midline laparotomy was performed to gain access to the abdominal cavity. A 18 mm long sharp incision was then made through the musculoperitoneal tissue on the left lateral abdominal wall below the epigastric vessel parallel with the midline incision. The injury site was immediately closed with four single sutures places equidistantly using 5-0 polypropylene threat (Prolene; Ethicon, Johnson & Johnson, Somerville, N.J., USA). Just before closing the abdomen, the animals received either mGluRgIa (MPEP 45.35 mM in vehicle) at a dose of 12.5 mg MPEP/kg (treatment group), or vehicle (0.9% NaC1, 10 w % dimethylacetatamide, ˜50 mM hydrochloride, vehicle group), by injecting the solution into the abdominal cavity. The midline laparotomy was then closed in two layers continuous suture using 5-0 polypropylene for the abdomen and 4-0 for the skin. The animals were kept on a heating pad during surgery until recovery from anaesthesia and thereafter returned to their respective cages. After one week the animals were sacrificed and the abdomen was reopened by U-shaped laparotomy with its base to the right. Formation of adhesions between the injury site and other uninjured structures within the abdominal cavity was macroscopically evaluated and quantified using the grading ‘0’ for absence of adhesions or ‘1’ for formed adhesions.

Results

4 of the 6 animals of the vehicle group had developed visible adhesions. 1 of the 6 animals of the treatment group had developed adhesions. The result may be illustrated as follows according to the grading explained above: Treatment group=100000, Vehicle group=111100.

This result shows that an mGluRgIa may be used for the treatment ofabnormal union of tissue.

Although the present invention has been described above with reference to specific illustrative embodiments, it is not intended to be limited to the specific form set forth herein. Any combination of the above mentioned embodiments should be appreciated as being within the scope of the invention. Rather, the invention is limited only by the accompanying claims and other embodiments than the specific above are equally possible within the scope of these appended claims.In the claims, the term ‘comprises/comprising’does not exclude the presence of other species or steps. Additionally, although individual features may be included in different claims, these may possibly advantageously be combined, and the inclusion in different claims does not imply that a combination of features is not feasible and/or advantageous. In addition, singular references do not exclude a plurality. The terms ‘a’, ‘an’, ‘first’, ‘second’ etc do not preclude a plurality.

1. A metabotropic glutamate receptor group I antagonist, or a pharmaceutically acceptable salt, prodrug or an optical isomer thereof, for use in the treatment or the prevention of abnormal union of tissue.

2. Metabotropic glutamate receptor group I antagonist according to claim 1, wherein said metabotropic glutamate receptor group I antagonist is an mGluR1 antagonist or an mGluR5 antagonist or a dual mGluR1/5 antagonist.

3. Metabotropic glutamate receptor group I antagonist according to claim 1, wherein said metabotropic glutamate receptor group I antagonist has an estimated drug suitability score which is greater than or equal to the drug suitability score for a comparative compound having a pharmaceutical effect other than affecting abnormal union of tissue due to antagonism of either or both of mGluR1 and mGluR5; or wherein said metabotropic glutamate receptor group I antagonist is having a pharmaceutical effect other than affecting abnormal union of tissue due to antagonism of either or both of mGluR1 and mGluR5; or wherein said metabotropic glutamate receptor group I antagonist is predominantly peripherally acting, having a ratio between the concentration in the brain and in the blood-plasma of less than 0.3.

4. Metabotropic glutamate receptor group I antagonist according to claim 1, wherein said abnormal union of tissue is intra abdominal adhesions.

5. Metabotropic glutamate receptor group I antagonist according to claim 4, wherein said intra abdominal adhesions is post-operative intra abdominal adhesions occurring after and due to abdominal surgery.

6-10. (canceled)

11. A method for the treatment or prevention of abnormal union of tissue, wherein an effective amount of a metabotropic glutamate receptor group I antagonist or a pharmaceutically acceptable salt, prodrug or an optical isomer thereof, or an effective amount of a pharmaceutical composition comprising a metabotropic glutamate receptor group I antagonist or a pharmaceutically acceptable salt, prodrug or an optical isomer thereof, is administered to a subject in need of such treatment or prevention.

12. Method according to claim 11, wherein said metabotropic glutamate receptor group I antagonist is an mGluR1 antagonist or an mGluR5 antagonist or a dual mGluR1/5 antagonist.

13. Method according to claim 11, wherein said metabotropic glutamate receptor group I antagonist has an estimated drug suitability score which is greater than or equal to the drug suitability score for a comparative compound having a pharmaceutical effect other than affecting abnormal union of tissue due to antagonism of either or both of mGluR1 and mGluR5; or wherein said metabotropic glutamate receptor group I antagonist is having a pharmaceutical effect other than affecting abnormal union of tissue due to antagonism of either or both of mGluR1 and mGluR5; or wherein said metabotropic glutamate receptor group I antagonist is predominantly peripherally acting, having a ratio between the concentration in the brain and in the blood-plasma of less than 0.3.

14. Method according to claim 11, wherein said abnormal union of tissue is intra abdominal adhesions.

15. Method according to claim 14, wherein said intra abdominal adhesions is post-operative intra abdominal adhesions occurring after and due to abdominal surgery.
